(19) United States
(12) Patent Application Publication
(10) Pub. No.: US 2023/0389908 A1
(43) Pub. Date: Dec. 7, 2023

(54) LOCKING RELEASE DEVICES, SYSTEMS, AND METHODS

(71) Applicant: BOSTON SCIENTIFIC SCIMED, INC., Maple Grove, MN (US)

(72) Inventors: Joshua Mark Inouye, Brooklyn Park, MN (US); Graham Krumpelmann, Stillwater, MN (US); James M. Anderson, Corcoran, MN (US)

(73) Assignee: BOSTON SCIENTIFIC SCIMED, INC., MAPLE GROVE, MN (US)

(21) Appl. No.: 18/204,652

(22) Filed: Jun. 1, 2023

Related U.S. Application Data
(60) Provisional application No. 63/348,286, filed on Jun. 2, 2022.

(51) Int. Cl. A61B 17/00

(52) U.S. Cl. CPC A61B 17/00234

(57) ABSTRACT

A latch locking and release system having a control knob operably coupled with a latch system, and a tubular latch release mechanism. The cross-section of the tubular latch release mechanism is shiftable between a locking configuration and a release configuration. In the locking configuration, the control knob maintains latches in the latch system in an engaged configuration, and is blocked from moving to allow the latches to be disengaged. In the release configuration, the control knob may be moved to allow the latches to be disengaged. The latch release mechanism and control knob may have further features preventing inadvertent actuation of the latch locking and release system to allow the latch system to be disengaged.

### CROSS-REFERENCE TO RELATED APPLICATIONS

This application claims the benefit of priority under 35 U.S.C. § 119 to U.S. Provisional Application No. 63/348,286, filed Jun. 2, 2022, the entire disclosure of which is hereby incorporated by reference herein for all purposes.

### FIELD

The present disclosure relates generally to the field of implantable medical devices. In particular, the present disclosure relates to devices, systems, and methods for releasing a latch engagement of a deployment device with respect to an implantable device.

### BACKGROUND

Various implantable medical devices are implanted with the use of a delivery/deployment device or system which delivers and implants the device at a treatment site. Some such implantable devices are implanted transluminally within the body with the use of a flexible elongate member extending between a control handle of the delivery/deployment system and the implantable device at a distal end thereof. The flexible elongate member must be sufficiently flexible to maneuver to the treatment site transluminally along a tortuous path. As the flexible elongate member is navigated to the treatment site, it is important that the implantable device remains engaged therewith. Moreover, it is important that the flexible elongate member remains operably engaged with the implantable device if the flexible elongate member is to be used in deploying the implantable device. For instance, some implantable devices and/or components thereof are rotated and/or axially translated by the flexible elongate member operably engaged therewith. Various latch systems have been used to engage a flexible elongate member with an implantable device. Improvements to devices, systems, and methods for operating such latch systems to maintain engagement between the flexible elongate member and the implantable device and/or to disengage the flexible elongate member and the implantable device would be welcome.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, a system for delivering and/or deploying an implantable device includes a flexible elongate member having a distal end with a delivery latch operably coupled therewith, and a proximal end opposite the distal end; a proximal control knob operably coupled with the proximal end of the flexible elongate member; a tubular flexible elongate member having a distal end with a lumen defined therein and a proximal end opposite the distal end; a distal control knob operably coupled with the proximal end of the tubular flexible elongate member; and a latch locking and release system shiftable between a locking configuration and a release configuration. In some aspects, the delivery latch is configured to engage a device latch on an implantable device, the lumen defined within the distal end of the tubular flexible elongate member is sized to extend over the delivery latch with a device latch engaged therewith to hold the delivery latch and the device latch in an engaged configuration; the latch locking and release system includes a tubular latch release mechanism sized, shaped, configured, and/or dimensioned to inhibit relative axial movement between the proximal control knob and the distal control knob when the latch locking and release system is in the locking configuration to maintain the delivery latch and the device latch in an engaged configuration; and in the release configuration, the latch release mechanism is sized, shaped, configured, and/or dimensioned to allow relative axial translation of the proximal control knob and the distal control knob along the longitudinal axis of the latch release mechanism to allow the delivery latch to be disengaged from the device latch.

In some embodiments, in the release configuration, a portion of one of the proximal control knob or the distal control knob extends into the interior of the latch release mechanism and moves relative to the other of the proximal control knob or the distal control knob to shift the tubular flexible elongate member with respect to the engaged latches to allow the delivery latch to be disengaged from the device latch. In some embodiments, the latch release mechanism is shiftable upon application of a radially-inwardly force thereto to shift the cross-sectional shape of the interior thereof to allow the one of the proximal control knob or the distal control knob to extend therein. In some embodiments, the latch release mechanism includes at least one projection riding over a corresponding ramp extending circumferentially on the exterior of the one of the proximal control knob or the distal control knob; the ramp decreases in depth from a first end to a second end thereof; when the projection is positioned over the first end of the ramp, the latch release mechanism and the one of the proximal control knob or the distal control knob are locked against relative translation with respect to each other; and when the projection is positioned over the second end of the ramp, the latch release mechanism and the one of the proximal control knob or the distal control knob are axially translatable with respect to each other.

In some embodiments, in the locking configuration, the latch release mechanism locks the tubular flexible elongate member against axially translating with respect to the flexible elongate member, thereby holding the delivery latch in engagement with the device latch within the lumen defined in the distal end of the tubular flexible elongate member; and in the release configuration, the latch release mechanism allows axial translation of the flexible tubular elongate member with respect to the flexible elongate member to allow the delivery latch to be disengaged from the device latch.

In some embodiments, the latch release mechanism is mounted on one of the proximal control knob or the distal control knob; and has an interior, when the latch locking and release system is in the release configuration, sized, shaped, configured, and/or dimensioned to receive the other of the proximal control knob or the distal control knob when the latch release mechanism and the other of the proximal control knob or the distal control knob are axially translated with respect to each other along a translation axis.

In some embodiments, the system includes a groove having a transverse leg extending transverse to the translation axis and an axial leg extending along the translation axis; and a projection extending transverse to the translation axis and into the groove, and preventing axial translation of the latch release mechanism and the other of the proximal control knob or the distal control knob when in the transverse leg of the groove, and allowing axial translation of the latch release mechanism and the other of the proximal control knob or the distal control knob when in the axial leg of the groove. In some embodiments, the system further includes an axial tab projection engaging an axial groove to prevent relative rotation of the projection into a position for movement along the axial leg of the groove.

In accordance with various principles of the present disclosure, a latch locking and release system is shiftable between a locking configuration maintaining a latch of the system engaged with a device latch operably coupled to an implantable device, and a release configuration allowing the latch of the system to be disengaged from the device latch. In some aspects, the system includes a control knob operably associated with the system latch; and a latch release mechanism shiftable between a locking configuration maintaining the control knob in a position maintaining engagement of the latches, and a release configuration allowing the control knob to release engagement of the latches; wherein the latch release mechanism is tubular about an axis and shiftable between a locking configuration blocking axial movement of the control knob with respect to the latch release mechanism and a release configuration allowing axial movement of the control knob with respect to the latch release mechanism.

In some embodiments, the tubular latch release mechanism has an interior sized, shaped, configured, and/or dimensioned to receive a portion of the control knob therein when in the release configuration, and to block the control knob from extending therein when in the locking configuration. In some embodiments, the latch release mechanism is shiftable upon application of a radially-inwardly force thereto to shift the cross-sectional shape of the interior thereof to allow the control knob to extend therein. In some embodiments, the latch release mechanism includes at least one projection riding over a corresponding ramp extending circumferentially on the exterior of the control knob; the ramp decreases in depth from a first end to a second end thereof; when the projection is positioned over the first end of the ramp the latch release mechanism and the control knob are locked against relative translation with respect to each other; and when the projection is positioned over the second end of the ramp the latch release mechanism and the control knob are axially translatable with respect to each other. In some embodiments, the interior of the latch release mechanism is changed as the projection rides from the first end of the ramp to the second end of the ramp from a cross-section sized, shaped, configured, and/or dimensioned blocking the control knob from extending into the interior of the latch release mechanism to a cross-section sized, shaped, configured, and/or dimensioned to allow a portion of the control knob to extend therein.

In some embodiments, the further includes a groove having a transverse leg extending transverse to the translation axis and an axial leg extending along the translation axis; and a projection extending transverse to the translation axis and into the groove, and preventing axial translation of the latch release mechanism and the control knob when in the transverse leg of the groove, and allowing axial translation of the latch release mechanism and the control knob when in the axial leg of the groove. In some embodiments, an axial tab projection engages an axial groove to prevent relative rotation of the projection into a position for movement along the axial leg of the groove.

In accordance with various principles of the present disclosure, a method of actuating a latch locking and release system includes changing the cross-section of a tubular latch release mechanism from a locking configuration sized, shaped, configured, and/or dimensioned to block axial translation of a control knob relative to the axis of the tubular latch release mechanism to a release configuration sized, shaped, configured, and/or dimensioned to allow axial translation of the control knob relative to the latch release mechanism along the axis of the tubular latch release mechanism.

In some aspects, the method further includes moving the control knob into the interior of the tubular latch release mechanism.

In some embodiments, the latch release mechanism and the control knob are locked against axial movement by engagement of a projection in a transverse leg of a groove, and the method further includes rotating the latch release mechanism and the control knob with respect to each other to move the projection into alignment with an axial leg of the groove to allow relative axial translation of the latch release mechanism and the control knob.

In some aspects, the method further includes squeezing the tubular latch release mechanism to change the cross-section thereof.

In some aspects, the method further includes rotating the latch release mechanism with respect to a ramp on the exterior of the control knob to change the cross-section of the latch release mechanism.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1A illustrates a perspective view of an example of an embodiment of a latch locking and release system formed in accordance with various principles of the present disclosure.

FIG. 1B illustrates a detail view of detail 1B of FIG. 1A.

FIG. 2 illustrates proximal perspective end view of an example of an embodiment of a latch locking and release system as in FIG. 1A.

FIG. 3A illustrates a cross-sectional view along line IIIA-IIIA of FIG. 2.

FIG. 3B illustrates an alternative configuration of an example of an embodiment of a latch locking and release system illustrated in FIG. 3A.

FIG. 4A illustrates a perspective view of a latch locking and release system as in FIG. 1A with the latch release mechanism in an initial activated configuration allowing the latch locking and release system to activate the latch locking and release system.

FIG. 4B illustrates a perspective view of a latch locking and release system as in FIG. 4A, but in a transition configuration allowing the latch locking and release system to transition into a configuration allowing disengagement of the latch locking and release system operatively associated therewith.

FIG. 4C illustrates a perspective view of a latch locking and release system as in FIG. 4A and FIG. 4B, but with the latch release mechanism in a configuration allowing disengagement of the latch locking and release system operatively associated therewith.

FIG. 5A illustrates a perspective view of another example of an embodiment of a latch locking and release system formed in accordance with various principles of the present disclosure.

FIG. 5B illustrates a detail view of detail 5B of FIG. 5A.

FIG. 6A illustrates a perspective view of a portion of a latch locking and release system as in FIG. 5A with the latch release mechanism in a locked configuration.

FIG. 6B illustrates a perspective view of a portion of a latch locking and release system as in FIG. 5A, but with the latch release mechanism in a configuration allowing disengagement of the latch locking and release system operatively associated therewith.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends. And “axial” generally refers to along a longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” is not limited to a circular cross-section. As used herein, a “free end” of an element is a terminal end at which such element does not extend beyond.

One trend in medical procedures includes moving from laparoscopic and open surgical procedures to miniaturized, minimally-invasive transcatheter or transluminal procedures in which a treatment site is accessed without cutting open the patient. Delivery and deployment systems have been developed to deliver implantable devices transluminally through a patient's body, such as transcatheterally through one or more delivery/deployment catheters. For instance, an implantable device may be delivered at the end of a flexible elongate member to a deployment site at which the implantable device may be deployed (e.g., implanted, engaged with another element, etc.). The flexible elongate member may be a stylet, a latch wire, a shaft, etc., (such terms being used interchangeably herein without intent to limit) capable of transluminal navigation to a treatment site, and optionally also deliverable through other tubular flexible elongate members. A delivery and deployment system used herewith includes a latch locking and release system selectively engageable with/disengageable from the implantable device to assure secure delivery of the implantable device at a distal end of the flexible elongate member. The latch locking and release system may also be used to assure secure engagement of the implantable device with an actuator system configured to deploy, adjust, or otherwise manipulate the implantable device before the delivery and deployment system is withdrawn. In some embodiments, the latch locking and release system includes a delivery latch at the end of the flexible elongate member which delivers the implantable device, and a device latch operably coupled with the implantable device (and which may be alternately referenced as a coupler). The delivery latch and the device latch are engageable with each other, and a hypotube may be translatable with respect to the engaged latches to maintain the latches in a latched configuration. Optionally, the hypotube acts as an actuator or driver which may manipulate, operate, actuate, control, etc., (such terms, and other grammatical forms thereof, being used interchangeably herein without intent to limit) the implantable device via the latches or via other components operatively associated with the implantable device. Optionally, one or more additional actuators may be operably coupled with the implantable device when in the latched configuration to allow further manipulation of the implantable device by the delivery and deployment system (e.g., to deploy the implantable device, to actuate one or more components or the device, etc.).

In accordance with various principles of the present disclosure, a latch release mechanism is operably associated with the delivery and deployment system to shift the latch locking and release system between an engaged and a disengaged configuration. More particularly, the latch release mechanism may have a locking configuration in which the latch release mechanism maintains the latch locking and release system in an engaged configuration in which the delivery latch and the device latch are maintained in an engaged configuration (e.g., with a hypotube over the delivery latch and the device latch to hold the latches in an engaged configuration). The latch release mechanism is actuatable to shift into a release configuration in which the latches of the latch locking and release system may be released from the engaged configuration and disengaged from each other.

In some embodiments, a latch locking and release system has a latch wire with a delivery latch and a hypotube translatable over the latch wire, such elements being translatable relative to each other between a latching configuration and a release configuration. In the latching configuration, the hypotube is positioned over the delivery latch and a device latch on an implantable device to maintain the latches in an engaged configuration. In the release configuration, the hypotube is in a position in which the latches are not both covered by the hypotube so that the latches may be disengaged from each other. Alternatively, in the release configuration of the hypotube, the hypotube is positioned with respect to the latch wire so that the delivery latch thereof is free to engage or to be engaged with the latch of an implantable device. The hypotube may then be shifted into the latching configuration to maintain the latches in an engaged configuration.

In accordance with various principles of the present disclosure, the latch release mechanism is actuatable with respect to a control knob to shift between a locking configuration and a release configuration. In the locking configuration, the latch release mechanism locks the control knob in position to maintain the latch locking and release system in the latching configuration. In the release configuration, the latch release mechanism is movable with respect to the control knob into a position allowing the control knob to shift the latch locking and release system into the release configuration.

In some embodiments, the latch release mechanism has a dual-locking arrangement preventing inadvertent shifting of the latch release mechanism from the locking configuration to the release configuration. For instance, the latch release mechanism and the control knob may be rotatable as well as axially translatable with respect to each other in shifting the latch release mechanism between the locking configuration and the release configuration. The dual-locking arrangement secures the latch release mechanism and control knob from inadvertent rotation as well as inadvertent axial translation with respect to each other. In some aspects, the latch release mechanism has a latch release collar with a configuration which is shiftable between a locking configuration and a release configuration. In some embodiments, interaction of the latch release collar with respect to the control knob shifts the latch release collar between the locking configuration and the release configuration.

Various embodiments of latch release mechanisms, systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

Turning now to the drawings, it will be appreciated that common features are identified by common reference elements and, for the sake of brevity and convenience, and without intent to limit, the descriptions of the common features are generally not repeated. For purposes of clarity, not all components having the same reference number are numbered. It will be appreciated that, in the following description, elements or components similar among the various illustrated embodiments with reference numbers greater than 100 are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

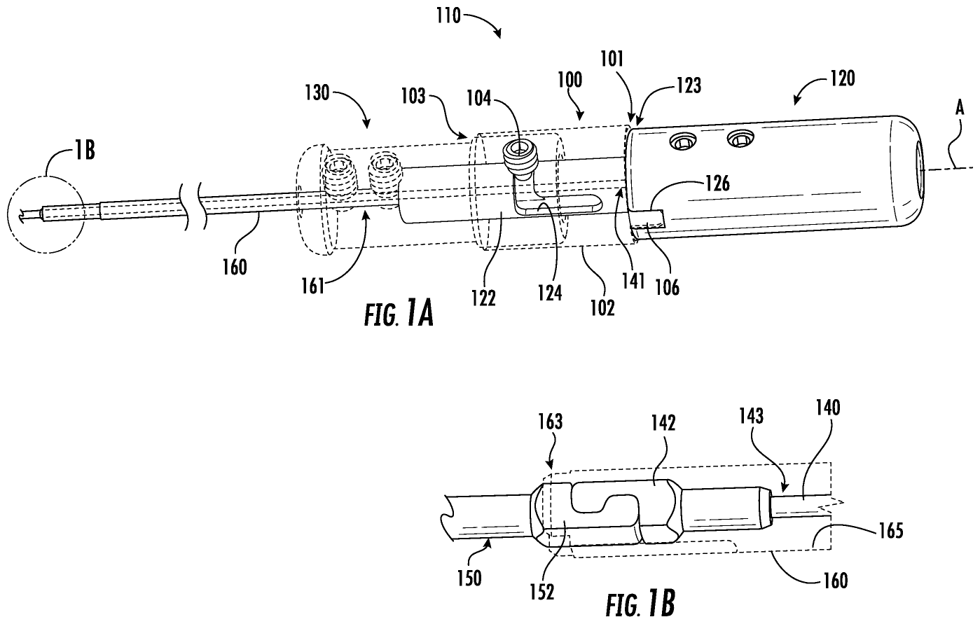

An example of an embodiment of a latch locking and release system 100 formed in accordance with various principles of the present disclosure is illustrated in FIG. 1A with respect to an example of an embodiment of a delivery and deployment system 110 (or at least a portion thereof). More particularly, the illustrated example of an embodiment of a latch locking and release system 100 includes a latch release mechanism 102 with a proximal end 101 positioned with respect to a distal end 123 of a proximal control knob 120 to inhibit or prevent relative movement therebetween (e.g., relative axial translation or rotation of the latch release mechanism 102 and the control knob 120), as discussed in further detail below. The latch release mechanism 102 may be mounted on or to another element of the delivery and deployment system 100, such as a distal control knob 130. For instance, in some embodiments, the distal end 103 of the latch release mechanism 102 may be fixed with respect to the distal control knob 130 to allow the proximal end 101 of the latch release mechanism 102 to be adjusted with respect to the proximal control knob 120 and optionally also with respect to the distal control knob 130. It will be appreciated that other relative configurations of a latch release mechanism 102 and one or more control knobs are within the scope and spirit of the present disclosure as well. For instance, instead of a proximal end 101 of the latch release mechanism 102 operably engaging a proximal control knob to inhibit or prevent relative movement therebetween, a distal end 103 of the latch release mechanism 102 may operably engage a distal control knob (and optionally be mounted on a proximal control knob) to inhibit or prevent relative movement therebetween. The latch locking and release system 100 is configured to shift between a locking configuration and a release configuration. In some embodiments, the latch release mechanism 102 is configured to prevent relative movement of the proximal control knob 120 and the distal control knob 130 when the latch locking and release system 100 is in the locking configuration, and to allow relative movement of the proximal control knob 120 and the distal control knob 130 when the latch locking and release system 100 is in the release configuration.

As may be appreciated with reference to FIG. 1A and the detail view thereof in FIG. 1B, the proximal control knob 120 is operably coupled with a proximal end 141 of a flexible elongate member 140 (such as along the distal end 123 of the proximal control knob 120). A delivery latch 142 is operably coupled to the distal end 143 of the flexible elongate member 140 in any suitable manner as known to those of ordinary skill in the art, such as riveting, welding, soldering, brazing, bonding (e.g., with adhesive or other material), crimping, interference fit, friction fit, etc., to guarantee a secure connection therebetween. The delivery latch 142 is configured to engage an implantable device 150, such as to deliver, deploy, actuate, move, etc. the implantable device 150. As illustrated in the detail view of FIG. 1B, the implantable device 150 may include a device latch 152 configured to be engaged with the delivery latch 142. Only a portion of an implantable device 150 is illustrated, as principles of the present disclosure are not limited to a particular implantable device 150. The implantable device 150 may be implantable into a patient's heart, such as into cardiac tissue (e.g., papillary muscle tissue), with respect to a cardiac valve (e.g., the mitral valve or the tricuspid valve), or with respect to the left atrial appendage, or with respect to any other anatomical structure.

In the illustrated example of an embodiment, the flexible elongate member 140 extends through the distal control knob 130 and a tubular flexible elongate member 160. The tubular flexible elongate member 160 extends from a proximal end 161 operably coupled to the distal control knob 130, to a distal end 163. The distal end 163 of the tubular flexible elongate member 160 may define a lumen 165 therein into which at least a portion of the delivery latch 142 (at the distal end 143 of the flexible elongate member 140) may be positioned, such as illustrated in the detail view of FIG. 1B. Optionally, the lumen 165 is sized to contain the delivery latch 142 and also the device latch 152 (or at least a portion thereof) therein. In some embodiments, the lumen 165 extends from the distal end 103 to the proximal end 161 of the tubular flexible elongate member 160. In some embodiments, the tubular flexible elongate member 160 may be configured as an actuator with a distal end 163 configured to operably engage the implantable device 150 to transmit movement of the tubular flexible elongate member 160 to the implantable device 150. In such case, movement (e.g., rotation) of the proximal control knob 120 causes movement (e.g., rotation) of the tubular flexible elongate member 160 which may also cause movement (e.g., rotation) of the implantable device 150 or a component thereof in various manners appreciated by those of ordinary skill in the art, the details of which do not limit the scope of the present disclosure.

The proximal end 161 of the tubular flexible elongate member 160 is operably coupled with the distal control knob 130 (e.g., via set screws) so that movement of the proximal control knob 130 controls movement of the tubular flexible elongate member 160, such as to axially translate the distal end 163 of the tubular flexible elongate member 160 with respect to the delivery latch 142 and the device latch 152. The flexible elongate member 140 and the tubular flexible elongate member 160 are axially translatable with respect to each other to allow the delivery latch 142 and the device latch 152 to shift between an engaged configuration (when within the lumen 165 in the tubular flexible elongate member 160, as illustrated in FIG. 1B) and a disengaged configuration (when not within the lumen 165). More particularly, when the delivery latch 142 and the device latch 152 are engaged to each other and within the lumen 165 of the tubular flexible elongate member 160 (or, in other words, the tubular flexible elongate member 160 is extended over the engaged delivery latch 142 and device latch 152), the delivery latch 142 and the device latch 152 generally cannot be disengaged from each other. Proximal withdrawal of the tubular flexible elongate member 160 to uncover the delivery latch 142 and the device latch 152, or distal advancement of the flexible elongate member 140 to move the delivery latch 142 out of the lumen 165 of the tubular flexible elongate member 160 allows the delivery latch 142 and the device latch 152 to be disengaged from each other.

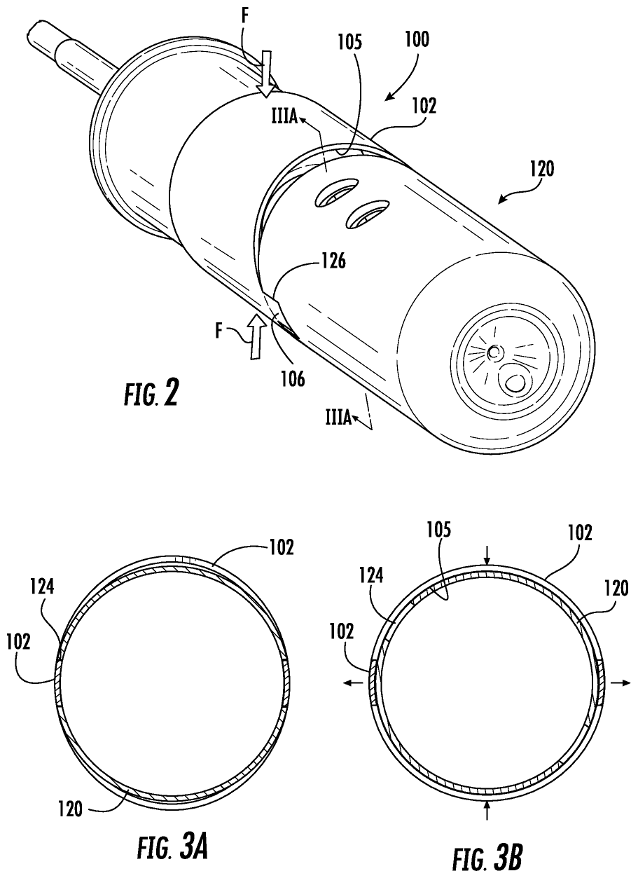

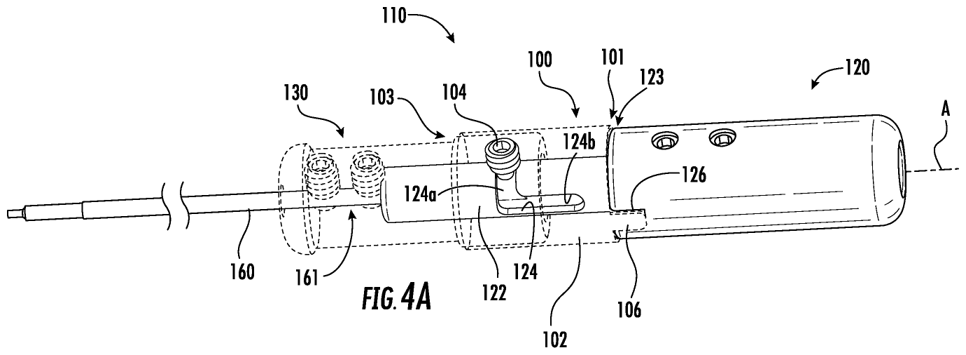

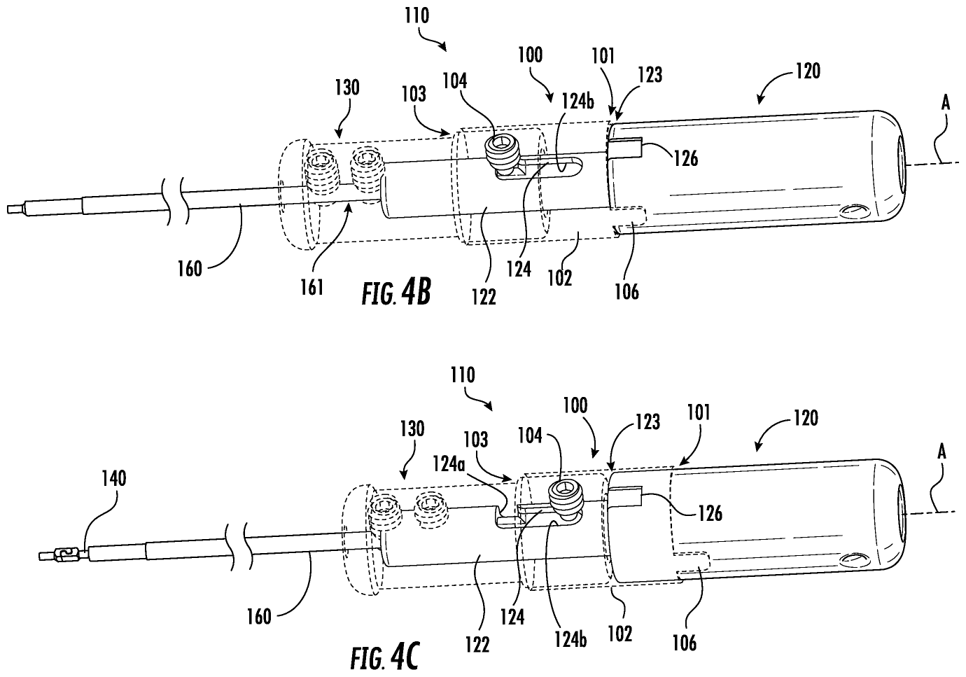

In accordance with various principles of the present disclosure, the latch locking and release system 100 is configured to shift between a locking configuration and a release configuration, such as illustrated in FIG. 2, FIG. 3A, FIG. 3B, FIG. 4A, FIG. 4B, FIG. 4C. In the locking configuration of the latch locking and release system 100, the delivery latch 142 and the device latch 152 are engaged with each other. For instance, in the locking configuration, the delivery latch 142 and the device latch 152 may be engaged and within the lumen 165 of the tubular flexible elongate member 160, and the tubular flexible elongate member 160 may be locked in position with respect to the flexible elongate member 140. In the illustrated example of an embodiment, the latch locking and release system 100 locks the proximal control knob 120 and the distal control knob 130 from moving with respect to each other, thereby maintaining the delivery latch 142 and the device latch 152 in engagement with each other. More particularly, the latch locking and release system 100 may lock the proximal control knob 120 and the distal control knob 130 with respect to each other so that the delivery latch 142 remains within the lumen 165 of the tubular flexible elongate member 160 and cannot be disengaged from the device latch 152. Even more particularly, the latch release mechanism 102 of the latch locking and release system 100 may be configured and positioned to prevent relative movement of the proximal control knob 120 and the distal control knob 130 to prevent relative movement of the tubular flexible elongate member 160 and the flexible elongate member 140 (with the delivery latch 142 at the distal end 143 thereof). In embodiments in which the latch release mechanism 102 is operably coupled with one of the control knobs 120, 130, the latch release mechanism 102 is inhibited or prevented from moving with respect to the other of the control knobs 120, 130 when the latch locking and release system 100 is in the locking configuration.

In the release configuration, the latch locking and release system 100 allows the delivery latch 142 and the device latch 152 to be disengaged (or to be engaged initially). For instance, in the release configuration, the delivery latch 142 and the device latch 152 are no longer within the lumen 165 within the tubular flexible elongate member 160 and may be disengaged from each other (or may be initially engaged with each other before being held in an engaged position within the lumen 165 of the tubular flexible elongate member 160). More particularly, in the release configuration, the latch release mechanism 102 may be shifted into a position and/or configured to allow relative movement between the flexible elongate member 140 and the tubular flexible elongate member 160 and/or relative movement between the proximal control knob 120 and the distal control knob 130. In embodiments in which the latch release mechanism 102 is operably coupled with one of the control knobs 120, 130, the latch release mechanism 102 is movable (e.g., axially translatable) with respect to the other of the control knobs 120, 130 when the latch locking and release system 100 is in the release configuration.

As may be appreciated with reference to the example of an embodiment of a latch locking and release system 100 illustrated in FIG. 2 in a locking configuration, the latch locking and release system 100 includes a tubular latch release mechanism 102 having a cross-section sized, shaped, configured, and/or dimensioned to block relative axial movement with respect to the proximal control knob 120. For instance, the interior 105 of the latch release mechanism 102 may have a cross-sectional shape which does not match the cross-sectional shape of the proximal control knob 120. Such difference in cross-sectional shapes of the latch release mechanism 102 and the proximal control knob 120 may be further appreciated with reference to the cross-sectional view of FIG. 3A along line IIIA-IIIA in FIG. 2. In the locking configuration, the latch release mechanism 102 blocks the proximal control knob 120 and the distal control knob 130 from moving relative to each other, thereby preventing relative movement between the flexible elongate member 140 and the tubular flexible elongate member 160, and thereby maintaining the delivery latch 142 and the device latch 152 in an engaged configuration (e.g., within the lumen 165 of the tubular flexible elongate member 160). For instance, at least a portion of the interior 105 of the latch release mechanism 102 is sized, shaped, configured, and/or dimensioned so that the proximal control knob 120 cannot move axially with respect to the latch release mechanism 102 (e.g., cannot move into the interior 105 of the latch release mechanism 102). For instance, in some embodiments, the cross-section of the proximal control knob 120 is generally circular whereas the cross-section of the latch release mechanism 102 is generally ovalized when in the locking configuration. In some embodiments, the periphery of the portion of the distal control knob 130 about which the latch release mechanism 102 is positioned includes narrowed regions (e.g., flats, or otherwise reduced diameter regions), or has an overall reduced diameter or proximal taper relative to distal portions of the distal control knob 130 to provide clearance for the latch release mechanism 102 to be configured to block movement of the proximal control knob 120 with respect to the latch release mechanism 102 and/or the distal control knob 130.

When a force F, such as a radially-inwardly directed force, is applied to the latch release mechanism 102 (as indicated in FIG. 2), the cross-sectional shape of the latch release mechanism 102 may be shifted to allow relative movement of the proximal control knob 120 with respect to the latch release mechanism 102 and/or the distal control knob 130. For instance, as illustrated in FIG. 3B, the latch release mechanism 102 may be shifted to have a generally round configuration. As may be appreciated with further reference to FIG. 3B, the cross-sectional shape of the latch release mechanism 102 may be shifted such that the cross-sectional shape of the interior 105 of the latch release mechanism 102 is shaped to receive the proximal control knob 120 therein. For instance, the interior 105 of the latch release mechanism 102 may be shifted into a configuration which does not block the proximal control knob 120 from being axially translated into the interior 105 of the latch release mechanism 102 along a longitudinal axis of the latch release mechanism 102 (generally along the axis A in FIG. 1A). In the example of an embodiment illustrated in FIG. 3B, the latch release mechanism 102 is shifted into a configuration with an interior 105 with a generally round cross-sectional shape having an inner diameter larger than the outer diameter of the proximal control knob 120. However, other configurations allowing relative movement of the latch release mechanism 102 and the proximal control knob 120 are within the scope and spirit of the present disclosure. It will be appreciated that in an embodiment in which the distal end 103 of the latch locking and release system 100 is operably coupled with the distal control knob 130, the latch locking and release system 100 may be flexible at the proximal end 101 thereof. In such embodiment, the distal end 103 of the latch locking and release system 100 may have a generally circular cross-section, or other shape conforming with the cross-sectional shape of the distal control knob 130.

When the latch locking and release system 100 is in the release configuration, the proximal control knob 120 may be axially and distally translated (e.g., advanced), such as to move into the interior 105 of the latch release mechanism 102. Alternatively or additionally, when the latch locking and release system 100 is in the release configuration, the distal control knob 130 may be axially and proximally translated (e.g., retracted), such as to move the latch release mechanism 102 over the proximal control knob 120. Such movement shifts the flexible elongate member 140 and the tubular flexible elongate member 160 with respect to each other to allow the delivery latch 142 and the device latch 152 to disengage.

In some embodiments, the latch locking and release system 100 is locked against inadvertent movement into the release configuration to protect against inadvertent disengagement of the delivery latch 142 and the device latch 152 (which may affect delivery and/or deployment and/or functioning of the associated implantable device 150). For instance, the latch locking and release system 100 may be locked to prevent inadvertent relative axial translation of the latch release mechanism 102 and the proximal control knob 120, even if the latch locking and release system 100 is in a release configuration with the latch release mechanism 102 configured to allow relative movement with respect to the proximal control knob 120 (e.g., into the interior 105 of the latch release mechanism 102). More particularly, in some embodiments, a transverse projection 104 (e.g., a radial projection such as, but not limited to, a set screw) may extend from the latch release mechanism 102 into a groove 124 in the proximal control knob 120, such as illustrated in FIG. 4A. In some embodiments, the projection 104 extends from the distal control knob 130 and the latch release mechanism 102 is operably coupled with the projection 104 via the distal control knob 130. In some embodiments, such as illustrated in FIGS. 4A-4C, the L-shaped groove 124 is in a distal, optionally narrower, extension 122 of the proximal control knob 120, although other configurations are within the scope and spirit of the present disclosure. In the illustrated example of an embodiment, in the locking configuration of the latch locking and release system 100, the projection 104 extends into an axially-transverse leg 124a of an L-shaped groove 124 (transverse to the axis A along which the proximal control knob 120 and the latch release mechanism 102 axially translate with respect to each other). In such position, the projection 104 and the axially-transverse leg 124a prevent relative axial translation of the latch release mechanism 102 and the proximal control knob 120 to maintain the flexible elongate member 140 and the tubular flexible elongate member 160 in positions with the delivery latch 142 and the device latch 152 within the lumen 165 of the flexible tubular elongate member 100. The projection 104 would have to be moved with respect to the axially-transverse leg 124a into alignment with an axially-extending leg 124b of the L-shaped groove 124 (such as by rotating the latch release mechanism 102 and the proximal control knob 120 with respect to each other) to move the projection 104 into alignment with the axially-extending leg 124b so that the latch release mechanism 102 and the proximal control knob 120 may be moved axially with respect to each other. As described above, and in further detail below, once the latch release mechanism 102 and the proximal control knob 120 move axially with respect to each other, the flexible elongate member 140 and the tubular flexible elongate member 160 may be shifted to a configuration allowing disengagement (or initial engagement) of the delivery latch 142 and the device latch 152.

In some embodiments, the latch release mechanism 102 may include at least one axial projection 106, such as a tab (e.g., a projection, finger, etc.), positioned along a side of the latch release mechanism 102 dimensioned (e.g., generally adjacent or along a side wall of the proximal control knob 120) such that the axial projection 106 is capable of engaging with an axial groove 126 in the proximal control knob 120 when the latch locking and release system 100 is in a locking configuration. In such configuration, as illustrated in FIG. 4A, the axial projection 106 and the axial groove 126 engage each other to prevent relative movement (rotational and optionally also translational) of the latch release mechanism 102 and the proximal control knob 120. As such, even if the latch release mechanism 102 is shifted into a configuration generally matching the configuration of the proximal control knob 120 as described above, the proximal control knob 120 may not be distally advanced into the interior 105 of the latch release mechanism 102 and the latch release mechanism 102 may not be proximally advanced over the proximal control knob 120. It will be appreciated that other configurations of tabs and grooves (e.g., not necessarily axially oriented, or in reverse positions with respect to the latch release mechanism 102 and the proximal control knob 120, etc.) are within the scope and spirit of the present disclosure.

As illustrated in FIG. 4B, squeezing of the latch release mechanism 102 to shift the cross-sectional shape of the proximal end 101 of the latch release mechanism 102 (such as described above) releases the tab 126 from engagement with the groove 126 to allow rotation of the projection 104 with respect to the L-shaped groove 124. The latch release mechanism 102 and the proximal control knob 120 may then be rotated with respect to each other (e.g., about the axis A) to move the projection 104 toward the axial leg 124b of the L-shaped groove 124, as illustrated in FIG. 4B. With the latch release mechanism 102 in a cross-sectional shape allowing the proximal control knob 120 to fit within the interior 105 of the latch release mechanism 102, and the projection 104 aligned with the axially-extending leg 124b, the latch release mechanism 102 and the proximal control knob 120 may be axially moved with respect to each other, as illustrated in FIG. 4C. And, as described above, relative axial movement of the latch release mechanism 102 and the proximal control knob 120 along the axis A allows relative movement of the flexible elongate member 140 and the tubular flexible elongate member 160 to allow the delivery latch 142 and the device latch 152 to disengage from each other (or to be engaged initially).

It will be appreciated that in some embodiments, instead of the latch release mechanism 102 being operably coupled with the distal control knob 130, the latch release mechanism 102 may be operably coupled with the proximal control knob 120. For instance, the proximal end 101 of the latch release mechanism 102 may be operably coupled with the proximal control knob 120 (such as in a manner as described with respect to coupling of the latch release mechanism 102 with respect to the distal control knob 130). An L-shaped groove with the longitudinally-extending leg thereof extending distally from the axially-transverse leg (i.e., in a mirror image configuration of the L-shaped groove 124 described above with respect to FIGS. 4A-4C) may be formed in the distal control knob 130 instead of in the proximal control knob 120. Optionally, the groove may be formed in a proximally-extending, optionally narrower extension of the distal control knob 130 (such as the reverse of the optional extension 122 of the proximal control knob 120). A projection may be provided in the latch release mechanism 102 and may operate generally as described above with respect to the example of an embodiment illustrated in FIGS. 4A-4D, but in a generally mirror-image direction.

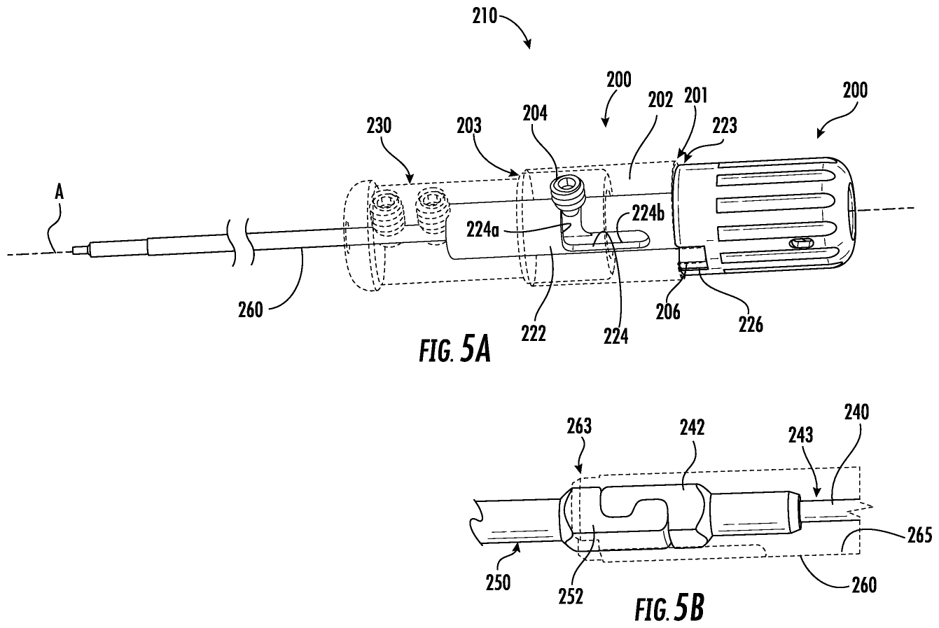

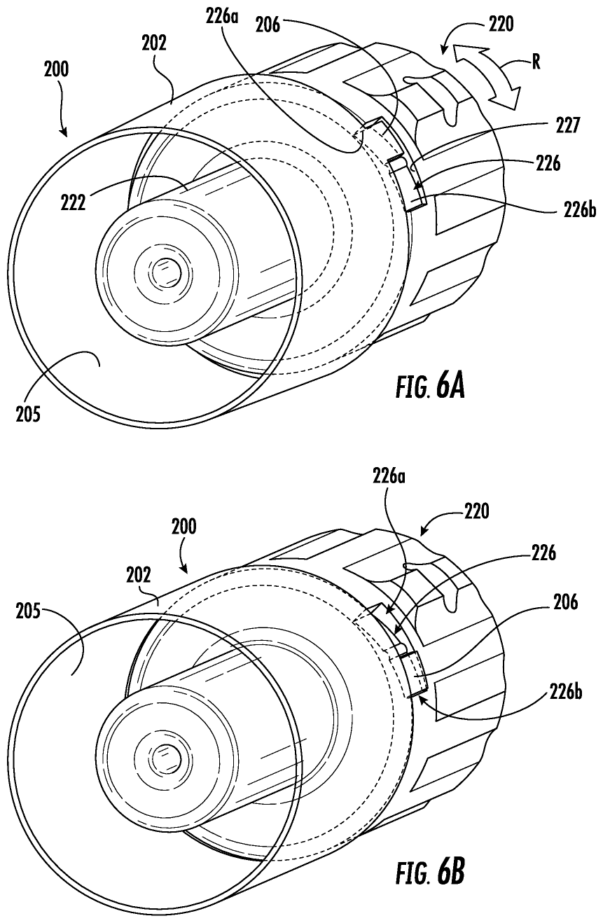

Another example of an embodiment of a latch locking and release system 200 with another example of an embodiment of a latch release mechanism 202 is illustrated in FIG. 5A, FIG. 5B, FIG. 6A, and FIG. 6B. It will be appreciated that elements in FIG. 5A, FIG. 5B, FIG. 6A, and FIG. 6B which are similar to elements in FIGS. 1A, 1B, 3A, 3B, and 4A-4C are numbered with similar reference numbers increased by 100, and reference is made to the above descriptions of similar elements and operations for the sake of brevity and convenience and without intent to limit. Similar to the above-described embodiment, the dimensions, shapes, and/or configurations of the latch release mechanism 202 and the proximal control knob 220 inhibit or prevent the latch release mechanism 202 and the proximal control knob 220 from axially translating relative to each other. For instance, similar to the above-described latch release mechanism 102 and proximal control knob 120, the latch release mechanism 202 and the proximal control knob 220 may have shapes which do not match and therefore prevent relative axial translation therebetween. More particularly, as may be appreciated with reference to FIG. 5A and FIG. 6A, the cross-sectional shape of the interior 205 of the latch release mechanism 202 may not match the cross-sectional shape of the exterior of the proximal control knob 220 so that the proximal control knob 220 cannot extend within the interior 205 of the latch release mechanism 202 (or, in other words, the latch release mechanism 202 cannot be extended over the proximal control knob 220). In a locking configuration of the latch locking and release system 200, the cross-sectional shape of the latch release mechanism 202 does not match the cross-sectional shape of the proximal control knob 220. In a release configuration of the latch locking and release system 200, the latch release mechanism 202 is sized, shaped, configured, and/or dimensioned to allow relative axial translation of the latch release mechanism 202 and the proximal control knob 220, such as allowing the proximal control knob 220 to extend into the interior 205 of the latch release mechanism 202 or the latch release mechanism 202 to be extended over the proximal control knob 220. Such relative movement of the latch release mechanism 202 and the proximal control knob 220 allows relative movement of the flexible tubular elongate member 200 and the engaged delivery latch 242 and device latch 252 into a position allowing disengagement (or initial engagement) of the delivery latch 242 and the device latch 252, such as described above.

In the latch locking and release system 200 illustrated in FIG. 5A, FIG. 6A, and FIG. 6B, instead of an axial projection 206 extending from a latch release mechanism 202 into an axial groove in the proximal control knob 220, at least one proximally-extending axial projection 206 on the latch release mechanism 202 extends over a corresponding ramp 226 extending circumferentially on the exterior of the proximal control knob 220. The axial projection 206 is positioned along a side of the latch release mechanism 202 dimensioned such that the axial projection 206 is capable of engaging the proximal control knob 220. The ramp 226 is deeper (i.e., the radial dimension of the proximal control knob 220 is smaller) at one circumferential end 226a thereof than another circumferential end 226b thereof, with the axial projection 206 in the deeper end 226a when the latch release mechanism 102 is in a locking configuration (in which the latch release mechanism 202 and the proximal control knob 220 are generally blocked from axially translating relative to each other). Once enough relative rotational force R is applied to the latch release mechanism 202 and the proximal control knob 220 (e.g., clockwise rotation of the latch release mechanism 202 and/or counterclockwise rotation of the proximal control knob 220 in the example of an embodiment illustrated in FIG. 6A), the axial projection 206 rides up the ramp 226. As the axial projection 206 rides up the ramp 226, because of the decreasing depth of the ramp 226 (and accompanying increase in the outer diameter of the proximal control knob 220), the axial projection 206 is moved radially outward from the axis A about which the latch release mechanism 202 and proximal control knob 220 are rotated with respect to each other. Such radially outward movement of the axial projection 206 causes the latch locking and release system 200 to shift into a release configuration. In particular, movement of the axial projection 206 causes deformation of the latch release mechanism 202. It will be appreciated that provision of two or more axial projections 206 (each in a corresponding ramp 226), circumferentially spaced apart (or at least a pair of diametrically opposed axial projection 206 and corresponding ramps 226) may facilitate the desired changing of the cross-sectional shape of the latch release mechanism 202 so as not to block axial translation thereof with respect to the proximal control knob 220. The latch release mechanism 202 may thereby be deformed from its locking configuration into a release configuration with the cross-sectional shape of the latch release mechanism 202 shifted into a release configuration allowing relative axial movement of the latch release mechanism 202 and the proximal control knob 220 to allow the delivery latch 242 and the device latch 252 to be disengaged. For instance, in the release configuration, the latch release mechanism 202 may be extended over the proximal control knob 220, and the distal end 263 of the tubular flexible elongate member 260 may be retracted from maintaining the delivery latch 242 and the device latch 252 in an engaged configuration therein. In other words, the proximal control knob 220 may extend into the interior 205 of the latch release mechanism 202 and the engaged delivery latch 242 and device latch 252 may be extended out from the lumen 265 in the tubular flexible elongate member 260 to be free to be disengaged from each other.

In some embodiments, as may be appreciated in the example of an embodiment illustrated in FIG. 6A and FIG. 6B, the deeper end 226a of the ramp 226 forms a proximal shoulder 227 in the proximal control knob 220 blocking axial movement of the axial projection 206, and thus the latch release mechanism 202, with respect to the proximal control knob 220. As such, the latch locking and release system 200 is in a locking configuration and the latch release mechanism 202 and the proximal control knob 220 are inhibited or prevented from moving axially with respect to each other. Once enough relative rotational force R is applied to the latch release mechanism 202 and the proximal control knob 220 (e.g., clockwise rotation of the latch release mechanism 202 and/or counterclockwise rotation of the proximal control knob 220 in the example of an embodiment illustrated in FIG. 6A), the axial projection 206 rides up the ramp 226. When the latch release mechanism 202 is rotated and the axial projection 206 reaches the second end 226b of the ramp 226, the shoulder 227 no longer blocks relative axial translation of the latch release mechanism 202. Also, as described above, movement of the axial projection 206 along the ramp 226 shifts the latch locking and release system 200 and the latch release mechanism 202 into a release configuration, allowing the latch release mechanism 202 and the proximal control knob 220 to shift with respect to each other. The latch release mechanism 202 and the proximal control knob 220 may be shaped and configured such that the axial projection 206 remains in the deeper end 226a of the ramp 226 (on the left side in FIG. 6A and FIG. 6B) unless a rotational force R is applied to one or both of the latch release mechanism 202 and the proximal control knob 220. The relative dimensions and configurations of the axial projection 206 and the ramp 226 are also selected such that the latch release mechanism 202 and the proximal control knob 220 are secured against inadvertent rotation with respect to each other. As such, in the locked configuration of the latch locking and release system 200, the latch release mechanism 202 and the proximal control knob 220 maintain the associated delivery latch 242 and device latch 252 in an engaged configuration. A delivery latch 242 on a distal end 243 of a flexible elongate member 240 having a proximal end 241 operably coupled to the proximal control knob 220 may thereby be inhibited or prevented from axial movement with respect to a tubular flexible elongate member 260 operably coupled at a proximal end 261 thereof to the distal control knob 230. When the delivery latch 242 is engaged with a device latch 252 and within a lumen 265 within a distal end 263 of the tubular flexible elongate member 260, such as illustrated in the detail view of FIG. 5A provided in FIG. 5B, such engaged configuration is maintained while the latch locking and release system 200 is in the locking configuration.

In some embodiments, the latch release mechanism 202 may include a transverse projection 204 and the proximal control knob 220 may include a corresponding L-shaped groove 224 which interact in a manner similar to the interaction between the projection 104 and L-shaped groove 124 described above with respect to FIGS. 4A-4C, reference being made thereto for the sake of brevity and convenience and without intent to limit. However, it will be appreciated that if the lumen in the tubular flexible elongate member of any of the above-described embodiments is dimensioned such that the engaged delivery latch and device latch cannot be moved proximally with respect to the tubular flexible elongate member (for instance, if the implantable device interferes with proximal retraction of the engaged latches further into the tubular flexible elongate member, and/or the lumen within the tubular flexible elongate member is sized, shaped, dimensioned, and/or configured to limit retraction of the latches therein, and/or otherwise), then proximal axial translation of the latch release mechanism with respect to the proximal control knob and/or distal axial translation of the latch release mechanism with respect to the proximal control knob to proximally retract the proximal control knob need not be inhibited, and the groove in the proximal control knob need not have a transverse leg. Similar principles (of lack of need of a groove with a transverse leg) may be applied with respect to a groove in a distal control knob as described above as may be appreciated by those of ordinary skill in the art.

It will be appreciated that the above-described order in which the latch release mechanism is operated may be reversed. For instance, the latch locking and release system may be in a release configuration to allow initial engagement of the delivery latch and the device latch, and the shifted to a locking configuration inhibiting or preventing disengagement of the latches. Moreover, as noted above, it will be appreciated that instead of the latch release mechanism engaging the proximal control knob, the latch release mechanism may engage the distal control knob. Operation of the latch release mechanism to shift between a locking configuration and a release configuration would be substantially the same as described above, except that the distal control knob would be shiftable proximally into the latch release mechanism (in contrast with the proximal control knob shifting distally into the latch release mechanism as described above).

It will be appreciated that features described with respect to one embodiment typically may be applied to another embodiment, whether or not explicitly indicated. The various features hereinafter described may be used singly or in any combination thereof. Therefore, the present invention is not limited to only the embodiments specifically described herein. It will further be appreciated that all apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. Various further benefits of the various aspects, features, components, and structures of a latch locking and release system such as described above, in addition to those discussed above, may be appreciated by those of ordinary skill in the art.

In view of the above, the foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system for delivering and/or deploying an implantable device, said system comprising:
a flexible elongate member having a distal end with a delivery latch operably coupled therewith, and a proximal end opposite the distal end;
a proximal control knob operably coupled with the proximal end of said flexible elongate member;
a tubular flexible elongate member having a distal end with a lumen defined therein and a proximal end opposite the distal end;
a distal control knob operably coupled with the proximal end of said tubular flexible elongate member; and
a latch locking and release system shiftable between a locking configuration and a release configuration;
wherein:
said delivery latch is configured to engage a device latch on an implantable device;
the lumen defined within the distal end of said tubular flexible elongate member is sized to extend over said delivery latch with a device latch engaged therewith to hold said delivery latch and the device latch in an engaged configuration;
said latch locking and release system includes a tubular latch release mechanism sized, shaped, configured, and/or dimensioned to inhibit relative axial movement between said proximal control knob and said distal control knob when said latch locking and release system is in the locking configuration to maintain said delivery latch and the device latch in an engaged configuration; and
in the release configuration, said latch release mechanism is sized, shaped, configured, and/or dimensioned to allow relative axial translation of said proximal control knob and said distal control knob along the longitudinal axis of said latch release mechanism to allow the delivery latch to be disengaged from the device latch.

2. The system of claim 1, wherein, in the release configuration, a portion of one of said proximal control knob or said distal control knob extends into the interior of said latch release mechanism and moves relative to the other of said proximal control knob or said distal control knob to shift said tubular flexible elongate member with respect to the engaged latches to allow said delivery latch to be disengaged from the device latch.

3. The system of claim 2, wherein said latch release mechanism is shiftable upon application of a radially-inwardly force thereto to shift the cross-sectional shape of the interior thereof to allow said one of said proximal control knob or said distal control knob to extend therein.

4. The system of claim 2, wherein:
said latch release mechanism includes at least one projection riding over a corresponding ramp extending circumferentially on the exterior of said one of said proximal control knob or said distal control knob;
said ramp decreases in depth from a first end to a second end thereof;
when said projection is positioned over the first end of said ramp, said latch release mechanism and said one of said proximal control knob or said distal control knob are locked against relative translation with respect to each other; and
when said projection is positioned over the second end of said ramp, said latch release mechanism and said one of said proximal control knob or said distal control knob are axially translatable with respect to each other.

5. The system of claim 1, wherein:
in the locking configuration, said latch release mechanism locks said tubular flexible elongate member against axially translating with respect to said flexible elongate member, thereby holding said delivery latch in engagement with the device latch within the lumen defined in the distal end of the tubular flexible elongate member; and
in the release configuration, said latch release mechanism allows axial translation of said flexible tubular elongate member with respect to said flexible elongate member to allow said delivery latch to be disengaged from the device latch.

6. The system of claim 1, wherein said latch release mechanism:
is mounted on one of said proximal control knob or said distal control knob; and
has an interior, when said latch locking and release system is in the release configuration, sized, shaped, configured, and/or dimensioned to receive the other of said proximal control knob or said distal control knob when said latch release mechanism and said other of said proximal control knob or said distal control knob are axially translated with respect to each other along a translation axis.

7. The system of claim 6, further comprising:
a groove having a transverse leg extending transverse to the translation axis and an axial leg extending along the translation axis; and
a projection extending transverse to the translation axis and into the groove, and preventing axial translation of said latch release mechanism and said other of said proximal control knob or said distal control knob when in the transverse leg of the groove, and allowing axial translation of said latch release mechanism and said other of said proximal control knob or said distal control knob when in the axial leg of the groove.

8. The system of claim 7, further comprising an axial tab projection engaging an axial groove to prevent relative rotation of said projection into a position for movement along the axial leg of the groove.

9. A latch locking and release system shiftable between a locking configuration maintaining a latch of said system engaged with a device latch operably coupled to an implantable device, and a release configuration allowing the latch of said system to be disengaged from the device latch, said system comprising:
a control knob operably associated with the system latch; and
a latch release mechanism shiftable between a locking configuration maintaining the control knob in a position maintaining engagement of the latches, and a release configuration allowing the control knob to release engagement of the latches;
wherein said latch release mechanism is tubular about an axis and shiftable between a locking configuration blocking axial movement of said control knob with respect to said latch release mechanism and a release configuration allowing axial movement of said control knob with respect to said latch release mechanism.

10. The latch locking and release system of claim 9, wherein said tubular latch release mechanism has an interior sized, shaped, configured, and/or dimensioned to receive a portion of said control knob therein when in the release configuration and to block said control knob from extending therein when in the locking configuration.

11. The latch locking and release system of claim 10, wherein said latch release mechanism is shiftable upon application of a radially-inwardly force thereto to shift the cross-sectional shape of the interior thereof to allow said control knob to extend therein.

12. The latch locking and release system of claim 9, wherein:
said latch release mechanism includes at least one projection riding over a corresponding ramp extending circumferentially on the exterior of said control knob;
said ramp decreases in depth from a first end to a second end thereof;
when said projection is positioned over the first end of said ramp, said latch release mechanism and said control knob are locked against relative translation with respect to each other; and
when said projection is positioned over the second end of said ramp, said latch release mechanism and said control knob are axially translatable with respect to each other.

13. The latch locking and release system of claim 12, wherein the interior of said latch release mechanism is changed as said projection rides from the first end of said ramp to the second end of said ramp from a cross-section sized, shaped, configured, and/or dimensioned blocking the control knob from extending into the interior of said latch release mechanism to a cross-section sized, shaped, configured, and/or dimensioned to allow a portion of said control knob to extend therein.

14. The latch locking and release system of claim 9, further comprising:
a groove having a transverse leg extending transverse to the translation axis and an axial leg extending along the translation axis; and
a projection extending transverse to the translation axis and into the groove, and preventing axial translation of said latch release mechanism and said control knob when in the transverse leg of the groove, and allowing axial translation of said latch release mechanism and said control knob when in the axial leg of the groove.

15. The system of claim 14, further comprising an axial tab projection engaging an axial groove to prevent relative rotation of said projection into a position for movement along the axial leg of the groove.

16. A method of actuating a latch locking and release system, said method comprising changing the cross-section of a tubular latch release mechanism from a locking configuration sized, shaped, configured, and/or dimensioned to block axial translation of a control knob relative to the axis of the tubular latch release mechanism to a release configuration sized, shaped, configured, and/or dimensioned to allow axial translation of the control knob relative to the latch release mechanism along the axis of the tubular latch release mechanism.

17. The method of claim 16, further comprising moving the control knob into the interior of the tubular latch release mechanism.

18. The method of claim 16, wherein the latch release mechanism and the control knob are locked against axial movement by engagement of a projection in a transverse leg of a groove, said method further comprising rotating the latch release mechanism and the control knob with respect to each other to move the projection into alignment with an axial leg of the groove to allow relative axial translation of the latch release mechanism and the control knob.

19. The method of claim 16, further comprising squeezing the tubular latch release mechanism to change the cross-section thereof.

20. The method of claim 16, further comprising rotating the latch release mechanism with respect to a ramp on the exterior of the control knob to change the cross-section of the latch release mechanism.
